(19) United States
(12) Patent Application Publication
(10) Pub. No.: US 2003/0072828 A1
(43) Pub. Date: Apr. 17, 2003

(54) Natural therapeutic composition for the treatment of wounds and sores

(76) Inventors: Dorothy Harrison, Strathmore (CA); Ernest Harrison, Strathmore (CA)

Correspondence Address: THOMAS E. MALYSZKO, SUITE 1500, 250 - 6 AVENUE, S.W., CALGARY, T2P 3H7, CA

(21) Appl. No.: 10/188,828

(22) Filed: Jul. 5, 2002

(30) Foreign Application Priority Data
Jul. 5, 2001 (CA) 2,352,674

(51) Int. Cl. A61K 35/78; A61K 31/07; A61K 31/355

(52) U.S. Cl. 424/776; 514/725; 514/458

(57) ABSTRACT

A therapeutic composition comprised of natural ingredients, namley quantities of Vitamin A, Vitamin E, Evening Primrose Oil and Mineral Oil, utilized with or without a suitable carrier, to safely and effectively sooth inflammation, ease pain, and promote the natural healing process of wounds and sores, particularly bed sores, and in the treatment of diseases, particularly psoriasis and ezcema.

More specifically, the invention, in one of its preferred embodiments, is a composition comprised of a combination of Vitamin A, Vitamin E, Evening Primrose Oil and Mineral Oil is specific quantities, to be applied topically or orally, particularly to bed sores, to reduce inflamation, ease pain, and assist the healing process of such wounds, sores and diseases. A quantity of Vitamin C and other natural ingredients may also be added to the composition with complimentary effects and with or without a carrier such as Petroleum Jelly in topical applications.

### FIELD OF THE INVENTION

This invention relates to a therapeutic composition for the treatment of wounds, sores, and diseases.

### DESCRIPTION OF PRIOR ART

Studies have shown that Vitamin A plays a role in the maintenance of skin; that Vitamin C helps fight infections, promotes the healing of cuts, abrasions and wounds and is required to maintain structural and functional integrity of cell walls and small blood vessels; that Vitamin E helps preened oxidation of fatty acids present in the membrane of all cells; that Evening Primrose Oil has anti-inflammatory and pain relieving effects; that Mineral Oil may be used to cleanse the skin; and that petroleum jelly may be used as a carrier for topical compositions.

The prior art relevant to this invention consists of various wound healing and anti-inflammatory compositions comprised of one or a few of the above ingredients and other herbal, vitamin and chemical mixtures.

It is known in the art that skin wound healing may be enhanced by the topical application of vitamin, herbal and/or chemical compositions to the affected area or through the ingestion of some such compositions. However, existing inventions inadequately address the need for a therapeutic composition that is all natural, provides fast pain relief, reduces inflammation and enhances the healing of wounds and sores.

U.S. Pat. No. 4,505,896 to J. E. Bernstein, filed Nov. 24, 1980, is a topical and oral method of treating lesions of acne vulgaris. It is comprised of a therapeutically effective amount of nicotine acid or nicotinamide. (The Bernstein patent does not claim use of Vitamin A, Vitamin C, Vitamin E, Evening Primrose Oil, Mineral Oil nor a petroleum carrier.)

U.S. Pat. No. 5,176,918 to J. L. Jones, filed Dec. 20, 1990, is a topical medicament, comprised of a dilute solution or sodium bicarbonate and a transdermal carrier for relieving pain from sunburn, minor burns and minor inflammatory diseases. (The Jones patent uses a transdermal carrier, typically glycerin, but does not claim use of Vitamin A, Vitamin C, Vitamin E, Evening Primrose Oil nor Mineral Oil.)

U.S. Pat. No. 5,240,945 to T. G. Warshaw, filed Feb. 18, 1002, is a method and composition for treating acne comprised of a lower alkyl nicotinate or methyl nicotinate. (The Warshaw patent utilizes a topically acceptable and compatible pharmaceutical carrier, such as petrolatum, but does not claim use of Vitamin A, Vitamin C, Vitamin E, Evening Primrose Oil nor Mineral Oil.)

U.S. Pat. No. 5,466,542 to B. A. Whittle, filed Feb. 28, 1992, is a pharmaceutical composition for the treatment of skin disorders comprised of extracts of herbs to provide an anti-inflammatory agent, an adrenocortical stimulant and a cortisol protecting agent. The herbs may be chosen from a group consisting of Pontentilla chinesis, Tribulus terrestris, Akebia trifolieata and select others. The solvent may be chosen from a group consisting of chloroform, methylene chloride, acetone, propanol, ethanol, methanol and select others. (The Whittle patent claims use of a carrier in form of a silica gel, but does not claim use of Vitamin A, Vitamin C, Vitamin E, Evening Primrose Oil nor Mineral Oil.) “Early Clinical Experience with Topical Collagen in Vascular Wound Care”, by J. Morris et al., published in 1994 by the Wound, Ostomy and Continence Nurses Society, discusses wound treatment for bilateral venous ulcers, including topical application of soap and water, hydrogen peroxide, saline solution and collagen. (The treatment mentioned in the article does not claim use of Vitamin A, Vitamin C, Vitamin E, Evening Primrose Oil, Mineral Oil nor a petroleum carrier.)

Canadian Patent No. 2,191,604 (U.S. Pat. No. 5,648,380) to A. Martin, filed May 22, 1995, laid open Jan. 11, 1996, is a therapeutic anti-inflammatory wound healing composition comprised of an anti-inflammatory agent and a wound healing composition. The wound healing composition is comprised of pyruvaqte, an antioxidant and a mixture of saturated and unsaturated fatty acids. The anti-inflammatory agent may be chosen from a group consisting of etodalc, Evening Primrose Oil, salsalate, cortisone and select others. The antioxidant may be chosen from a group consisting of all forms of Vitamin A, Vitamin C, Vitamin E and mixtures thereof. The composition is also comprised of a pharmaceutically acceptable carrier. This may be distinguished from this invention by Mineral Oil in the claims.

Canadian Patent No. 2,192,603 (U.S. Pat. No. 5,663,208) to A. Martin, filed May 22, 1995, laid open Feb. 8, 1996, is an anti-fungal wound healing composition with anti-fungal agent comprised of lactic and sorbic acid, terconazole or select others.

U.S. Pat. No. 5,652,274 to A. Martin, filed May 22, 1995, is a therapeutic wound healing composition and method of preparation. The wound healing composition is comprised of pryuvate, and antioxidant and a mixture of saturated and unsaturated fatty acids. This may be distinguished from this invention by Mineral Oil in the claims.

U.S. Pat. No. 5,674,912 to A. Martin, filed May 22, 1995, is a sun-screen wound healing composition and method of preparation. The sun-screen agent may be selected from a group consisting of titanium dioxide, urea, oxybenzone and select others.

(All of the Martin patents claim use of Vitamin A, Vitamin C and/or Vitamin E, as an antioxident, Evening Primrose Oil as an anti-inflammatory agent and a pharmaceutically acceptable carrier. However, the Martin patents require that an agent and a wound healing composition, they must be comprised of pyruvate, an antioxidant, and a mixture of saturated and unsaturated fatty acids, must be used together. The Martin patents do not claim use of Mineral Oil)

U.S. Pat. No. 5,916,573 to S. M. Spiers et al., filed Aug. 1996 is a topical treatment of the skin with a grape-seed oil composition. The composition is comprised of grape-seed oil, a hydrating agent that may be chosen from a group consisting of Vitamin E oil, Primrose oil and select others, an amino acid and deionized water. (The Spiers patent does not claim use of Vitamin A, Vitamin C nor Mineral Oil.)

U.S. Pat. No. 6,107,349 to P. Mantynen, filed Apr. 16, 1998, is a method and composition for treating psoriasis. The method is comprised of oral ingestion by a patient of Vitamin E, Evening Primrose Oil, folic acid and B-complex vitamins. (The Mantynen patent is not topical and does note claim use of Vitamin A, Vitamin C, Mineral Oil nor a petroleum carrier.)

Canadian Patent No. 2,329,160 to S. Leibovich (U.S. Pat. No. 6,187,822), filed in the United States on Jun. 11, 1998, laid open in Canada on Dec. 16, 1999, is a wound treatment through inhibition of adenoside diphosphate ribosyl transferase. The inhibitor is comprised from a group consisting of Vitamin K1, K2, K3, K4 and select others. The composition is further comprised of a pharmaceutically acceptable carrier. An anti-inflammatory agent may also be used that is comprised of Evening Primrose Oil. (The Leibovich patent does not claim use of Vitamin A, Vitamin C, Vitamin E nor Mineral Oil.)

U.S. Pat. No. 6,203,805 to D. F. Collins et al., filed Nov. 10, 1998, is a topical composition containing whey proteins, Vitamin A, Vitamin C, Vitamin E, to improve the resiliency of the skin. (The Collins patent does not claim use of Evening Primrose Oil, Mineral Oil nor a petroleum carrier.)

German Patent No. 19800982 to F. Karl-Heinz, filed Jan. 14, 1999, is an ointment containing ethereal oil, liposomes and vitamins for treating neruoodermatitis, psoriasis and eczema. The medical ointment base is comprised of ingredients selected from a group comprised of Vaseline, beeswax, paraffin or select others. The active ingredient used in conjunction with the base may be chosen from a group consisting of vitamins, liposome and ethereal. (The Karl-Heinz patent does not claim use of Evening Primrose Oil nor Mineral Oil.)

U.S. Pat. No. 6,183,761 to D. L. Bissett, filed Mar. 12, 1999, is a composition for regulating skin appearance. The topical composition is comprised of a Vitamin B3 compound, a polycyclic compound, flavanones, chalcones, isoflavones, coumarins, chromones, dicomarols, chromanols, sterols, selected others and mixtures thereof. (The Bissett patent does not claim use of Vitamin A, Vitamin C, Vitamin E, Evening Primrose Oil, Mineral Oil nor a petroleum carrier.)

Canadian Patent No. 2,285,818 to J. C. Liu et al, filed Oct. 12, 1999, laid open Apr. 13, 2000, is a composition to enhance skin permeation of topical skin agents. The composition is comprised of an active agent that may be chosen from a group consisting of hydrophobic and hydrophilic active agents and a polymeric emulsifier. The hydrophobic and hydrophilic agents may be chosen from a group consisting of Vitamin A, Vitamin C, Vitamin E, wound healing promoters, sun-screens, fragrances and select others. (the Liu patent does not claim use of Evening Primrose Oil, Mineral Oil nor a petroleum carrier.)

U.S. Pat. No. 6,248,763 to R. Scivoletto, filed Oct. 12, 1999, is a composition for treating skin conditions, comprising of methyl nicotinate, aloe vera gel, Vitamin E, glycerin, silk amino acids and select others. (The Scivoletto patent does not claim use of Vitamin A, Vitamin C, Evening Primrose Oil nor Mineral Oil.)

U.S. Pat. No. 6,187,743 to E. T. Obi-Talbot, filed Dec. 10, 1999, is a composition and method for enhancing wound healing. The method is comprised of a composition of bovine collagen Type I and aipha-tocopherol in a mixture which may be combined with vitamins. (The Obi-Talbot patent does not claim use of Evening Primrose Oil, Mineral Oil nor a petroleum carrier.)

As disclosed above, a number of wound and disease treating compositions exist that are topical and ingested, and are composed of herbs, chemicals, and vitamins. However, none of the prior art discloses an invention that utilizes a topically and orally administered natural therapeutic composition for the treatment of diseases and wounds comprised of a mixture of Vitamin A, Vitamin E, Evening Primrose Oil and Mineral Oil.

### SUMMARY OF THE INVENTION

This invention relates to a therapeutic composition for the treatment of wounds and diseases that is comprised of natural ingredients.

From personal experience we have discovered the following traits associated with the specific components of the invention:

Vitamin A topically applied to the skin, particularly on the face, results in improved appearance of the skin;

Vitamin C acts like natural Aspirin® by combating pain and inflammation. It is known to help arthritis;

Vitamin E topically applied to scalds results in reduced pain and improved healing of the wound;

Evening Primrose Oil topically applied to the face results in a healthier appearance of the skin. Used on wounds, i.e. after a gall bladder surgery, applied daily for six weeks, results in advanced healing of the wound. It may also be used to treat chronic fatigue syndrome;

Mineral Oil topically applied to severe sunburn may result in easement of pain and reduced inflammation of the wound;

Myrrah has antiseptic and astringent properties that may reduce inflammation and infection, thereby promoting healing of healthy tissue;

Olive Oil aids in dry skin problems and helps to clean and heal wounds, ulcers, sores, abrasions, and sunburn; and,

Peppermint Extract topically applied, particularly to the head, results in reduced pain in, for example, cases of headache, sinus and earache.

Using our knowledge of and experience with the above ingredients, we experimented with various quantities of all of the ingredients combined into one composition, with several slight variants for specific indications. We discovered that the ingredients work well together and complemented and enhanced the restorative and healing effects of each other when combined, particularly in the proportions disclosed in the formulae of the various embodiments of the invention.

The invention is comprised of four (4) main elements and may be used topically or internally:

(a) Vitamin A

(b) Vitamin E

(c) Evening Primrose Oil

(d) Mineral Oil

In addition to the elements listed above, small amounts of Vitamin C and other natural ingredients such as Olive Oil, Peppermint Extract and Myrrah may be present in some embodiments. Additionally, some embodiments may use a suitable carrier, such as Petroleum Jelly, for topical application. The amount of carrier used can be varied to regulate the consistency of the composition. In some indications a thicker composition is more desirable.

### DESCRIPTION OF THE PREFERRED EMBODIMENTS

The invention, in a first preferred embodiment, is a topical or oral composition comprised of the following ingredients in the following proportions by volumes:

| | |
|---|---|
| Vitamin A | 0.5-2.5% |
| Vitamin E | 35-45% |
| Evening Primrose Oil | 45-55% |
| Mineral Oil | 4.5-6.5% |

This embodiment of the invention is effective as a therapeutic agent for the treatment of bedsores, earache, headache, and ezcema of and around the eye when applied on or proximal to the affected areas.

### EXAMPLE

A test of the preferred embodiment of the invention was performed in Feb. 2001 using the following composition of ingredients:

| | | |
|---|---|---|
| Vitamin A | 0.5 | ml |
| Vitamin E | 15 | ml |
| Evening Primrose Oil | 20 | ml |
| Mineral Oil | 2.0 | ml |

The patient was suffering from severe bedsores. We topically applied the composition of the preferred embodiment of the invention to the patient's wounds daily with startling success. The attending physicians and care workers were also very impressed with the results. The patient admitted that the pain diminished instantly upon the application of the composition that is the preferred embodiment of this invention and after five days of daily use, the sores were not painful at all. The inflammation of the wound greatly diminished and the wound appeared to be healing from the inside to the outside, at a rate much more rapid than normally associated with such sores. We have also tried the invention on itches, scalds, cold sores, sore throats, and bums with similar results.

In a second and further embodiment, the present invention provides a topical composition comprised of the same proportions of ingredients as in the first embodiment above, but using Petroleum Jelly as a carrier.

We have used the second embodiment in the successful treatment of psoriasis, body itch, insect bites, bums and scalds, and dry skin on the hands and face when it was applied on or proximal to the affected areas.

In a third and further embodiment, the present invention provides a topical composition comprised of the following ingredients in the following proportions by volumes:

| | |
|---|---|
| Vitamin A | 0.5-2.0% |
| Vitamin E | 30-45% |
| Evening Primrose Oil | 45-55% |
| Mineral Oil | 4-6% |
| Olive Oil | 4-6% |

in a suitable carrier, such as Petroleum Jelly.

We have used the third embodiment in the successful treatment of psoriasis, body itch, insect bites, dry skin on the face and hands, bums and scalds by applying it on or proximal to the affected areas. In one example of the third embodiment used topically for such treatment the composition had the following ingredients:

| | | |
|---|---|---|
| Vitamin A | 0.5 | ml |
| Vitamin E | 15 | ml |
| Evening Primrose Oil | 20 | ml |
| Mineral Oil | 2.0 | ml |
| Olive Oil | 2.0 | ml |

In a fourth and further embodiment, the present invention provides a topical composition comprised of the following ingredients in the following proportions by volumes:

| | |
|---|---|
| Vitamin A | 0.5-2% |
| Vitamin C | 2-3.5% |
| Vitamin E | 30-40% |
| Evening Primrose Oil | 45-55% |
| Mineral Oil | 3.5-6.5% |
| Myrrah | 0.5-1.5% |
| Olive Oil | 3.5-6.5% |

in a suitable carrier, such as Petroleum Jelly.

We have used the fourth embodiment in the successful treatment of some types of psoriasis and some types of ezcema by applying it on or proximal to the affected area. In one example of the fourth embodiment used topically for such treatment the composition had the following ingredients:

| | | |
|---|---|---|
| Vitamin A | 0.5 | ml |
| Vitamin C | 1.0 | ml |
| Vitamin E | 15 | ml |
| Evening Primrose Oil | 20 | ml |
| Mineral Oil | 2.0 | ml |
| Myrrah | 0.4 | ml |
| Olive Oil | 2.0 | ml |

In a fifth and further embodiment, the present invention provides a topical or oral composition comprised of the following ingredients in the following proportions by volumes:

| | |
|---|---|
| Vitamin A | 0.5-2% |
| Vitamin C | 2-3.5% |
| Vitamin E | 30-40% |
| Evening Primrose Oil | 45-55% |
| Mineral Oil | 3.5-6.5% |
| Myrrah | 0.5-1.5% |
| Olive Oil | 3.5-6.5% |
| Peppermint Extract | 0.5-1.5% |

in a suitable carrier, such as Petroleum Jelly, for topical applications.

We have used the fifth embodiment in the successful treatment of headaches and migraine. In one example of the fifth embodiment used topically by applying it on or proximal to the affected areas for such treatment, the composition had the following ingredients:

| | | |
|---|---|---|
| Vitamin A | 0.5 | ml |
| Vitamin C | 1.0 | ml |
| Vitamin E | 15 | ml |
| Evening Primrose Oil | 20 | ml |
| Mineral Oil | 2.0 | ml |
| Myrrah | 0.4 | ml |
| Olive Oil | 2.0 | ml |
| Peppermint Extract | 0.4 | ml |

As demonstrated, the invention provides a new and effective therapeutic composition comprised of all natural ingredients, for the treatment of wounds, sores and diseases.

While the invention has been described in detail and with reference to specific embodiments thereof, it will be apparent to one skilled in the art that various changes and modifications can be made therein without departing from the spirit and scope thereof.

We claim:

1. A topical compound comprising:
Vitamin A;
Vitamin E;
Evening Primrose Oil; and
Mineral Oil.
